(19) United States
(12) Patent Application Publication
(10) Pub. No.: US 2012/0101512 A1
(43) Pub. Date: Apr. 26, 2012

(54) DEBRIDEMENT HAND TOOL

(76) Inventors: Christopher Brian Locke, Bournemouth (GB); Timothy Mark Robinson, Basingstoke (GB); Richard Paul Mormino, San Antonio, TX (US); Eric Woodson Barta, San Antonio, TX (US); Richard Marvin Kazala, San Antonio, TX (US)

(21) Appl. No.: 13/279,029

(22) Filed: Oct. 21, 2011

Related U.S. Application Data
(60) Provisional application No. 61/405,577, filed on Oct. 21, 2010.

(51) Int. Cl. A61B 17/32

(52) U.S. Cl. 606/169

(57) ABSTRACT

The disclosed embodiments include tools for aiding a user in debriding necrotic tissue. For example, in one embodiment, a debridement tool is disclosed that includes a housing having an interior space. A motor and a power source are disposed within the interior space of the housing. The power source is operable to provide power to the motor. A blade is coupled to a first end of the housing. The blade is operable to vibrate when the motor is powered. The debridement tool further includes a lighting element disposed within the housing and beneath a cutting edge of the blade. The lighting element is operable to illuminate the tissue site. In some embodiments, the lighting element operates at a particular wavelength that assists in distinguishing between necrotic tissue and healthy tissue.

### CROSS-REFERENCE TO RELATED APPLICATIONS

This application claims the benefit of U.S. Provisional Application No. 61/405,577, filed Oct. 21, 2010, which is hereby incorporated by reference.

### BACKGROUND OF THE INVENTION

Debridement refers to the removal of dead, damaged, or infected tissue to improve the healing potential of the remaining healthy tissue. Several factors make proper debridement difficult such as poor lighting, difficult wound locations, immobile patients, environmental constraints, and the use of improper debridement tools. In addition, the action of cutting away the debrided eschar is especially difficult for outside of a hospital or controlled medical environment because the blade, often a traditional pointed blade, may cut into healthy tissue and cause extensive bleeding. Therefore, this fear may cause some users to debride “lightly”. This can often lead to infections due to eschar remaining in the wound after cleaning and redressing.

### SUMMARY

The disclosed embodiments provide solutions to problems associated with existing debridement tools. For example, in one embodiment, a debridement tool for debriding tissue from a tissue site is presented. The debridement tool includes a housing having an interior space. A motor and a power source are disposed within the interior space of the housing. The power source is operable to provide power to the motor. A blade is coupled to a first end of the housing. The blade is operable to vibrate when the motor is powered. The debridement tool further includes a lighting element disposed within the housing and beneath a debridement edge of the blade. The lighting element is operable to illuminate the tissue site. In some embodiments, the lighting element operates at a particular wavelength that assists a user in distinguishing between necrotic tissue and healthy tissue.

The disclosed embodiments also provide several types of blades that may be used to debride necrotic tissue. For instance, in one embodiment, a curette-style blade for use with a debridement tool is disclosed. The curette-style blade includes an extension member having a first end and a second end, an attachment member for coupling the first end of the blade to the debridement tool, and a circular debridement member attached to the second end of the extension member. The circular debridement member is operable to debride necrotic tissue from a tissue site.

In another embodiment, a ribbon-style blade for use with a debridement tool is disclosed. The ribbon-style blade includes an elongated member forming a shape with an interior space, one or more support structures located within the interior space and connected to an interior perimeter of the elongated member for supporting the shape of the elongated member, and an attachment member for coupling the blade to the debridement tool. The elongated member is operable to assist a user in debridement of necrotic tissue.

Other objects, features, and advantages of the illustrative embodiments will become apparent with reference to the drawings and detailed description that follow.

### BRIEF DESCRIPTION OF THE DRAWINGS

FIG. 1 illustrates a debridement tool operated by a user to debride necrotic tissue at a tissue site in accordance with one embodiment;

FIG. 2 illustrates a perspective view of a debridement tool in accordance with one embodiment;

FIG. 3 illustrates the interior components of a debridement tool in accordance with one embodiment;

FIG. 4 illustrates a perspective view of a debridement tool in accordance with another embodiment;

FIG. 5 illustrates a front perspective view of the debridement tool of FIG. 4;

FIG. 6 illustrates components of the debridement tool of FIG. 4 in accordance with one embodiment;

FIG. 7 illustrates a debridement blade that may be used with a debridement tool in accordance with one embodiment;

FIG. 8 illustrates another debridement blade that may be used with a debridement tool in accordance with the disclosed embodiments;

FIG. 9 illustrates yet another debridement blade that may be used with a debridement tool in accordance with the disclosed embodiments;

FIG. 10 illustrates a debridement according to an illustrative embodiment; and

FIGS. 11-33 illustrate multiple embodiments of debridement blades or tips configured to remove tissue and configured to be used with a debridement tool.

### DETAILED DESCRIPTION OF ILLUSTRATIVE EMBODIMENTS

In the following detailed description of several illustrative embodiments, reference is made to the accompanying drawings that form a part hereof, and in which is shown by way of illustration specific embodiments in which the invention may be practiced. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments are defined only by the appended claims.

The terms “debride”, “debriding”, and “debridement” as used herein refers to the removal of necrotic tissue to improve the healing potential of the remaining healthy tissue. The term “necrotic tissue” as used herein refers to dead, damaged, or infected tissue. Although debridement of necrotic tissue may sometimes require an incision or cut be made, the disclosed embodiments may also be used to debride necrotic tissue without requiring that any incisions be made. The term “tissue site” as used herein refers to a wound or defect located on or within any tissue, including but not limited to, bone tissue, adipose tissue, muscle tissue, neural tissue, dermal tissue, vascular tissue, connective tissue, cartilage, tendons, or ligaments. Finally, the term “blade” as referenced herein refers to a component of the debridement tool that comes in contact with necrotic tissue and is capable of being used for debriding necrotic tissue from a tissue site. The term “blade” does not imply or infer that the blade is necessarily sharp or capable of cutting.

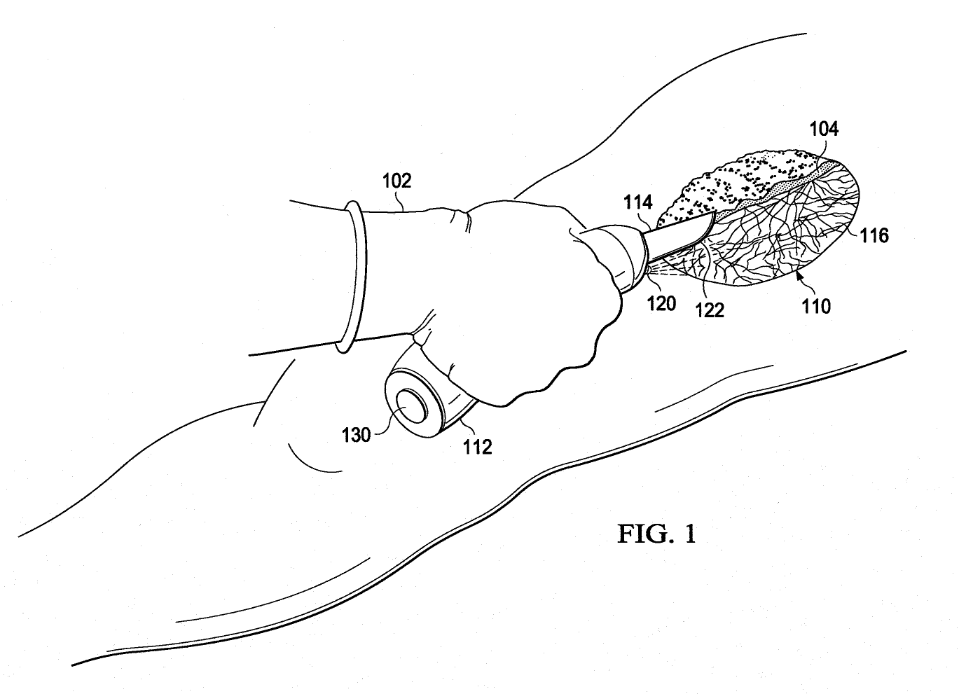

FIGS. 1-3 are related to one embodiment of a debridement tool 100 for debriding necrotic tissue. Beginning with FIG. 1, a perspective view of a user 102 using the debridement tool 100 to debride the necrotic tissue 104 at a tissue site 110 is presented. The debridement tool 100 comprises a housing 112. The housing 112 serves as a handle, as shown in FIG. 1, for enabling the user 102 to grip and use the debridement tool 100. The user 102 may be a caregiver, such as a nurse or doctor, or may be a patient performing the debridement procedure on him or herself. In one embodiment, the debridement tool 100 is designed specifically for use in a home-care environment where optimal lighting and conditions are not present.

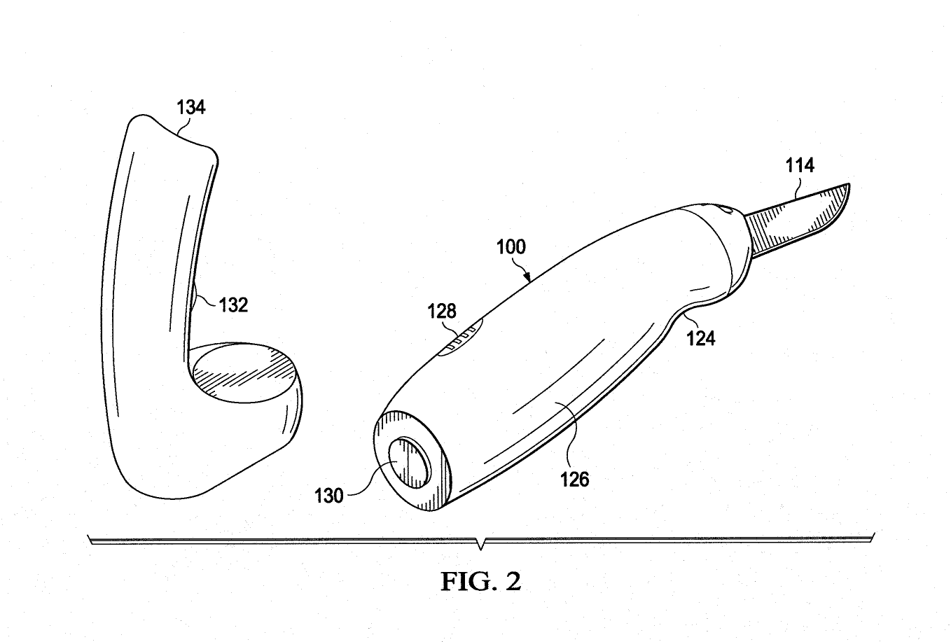

Referring to FIG. 2, in certain embodiments, the housing 112 may be manufactured to provide an ergonomically shaped handle so as to provide the user 102 with a comfortable grip when using the debridement tool 100. The housing 112 may be made of any material, such as plastic or metal. The housing 112 may be formed by an injection molding process or any other suitable technique. In one embodiment, the housing 112 may be formed as a single unit having an interior space for housing components of the debridement tool 100. Alternatively, the housing 112 may be formed by coupling together two or more separate pieces. The interior of the housing 112 may also include support mounts, brackets, and/or other attachment means for coupling one or more of the internal components of the debridement tool 100 to the housing 112.

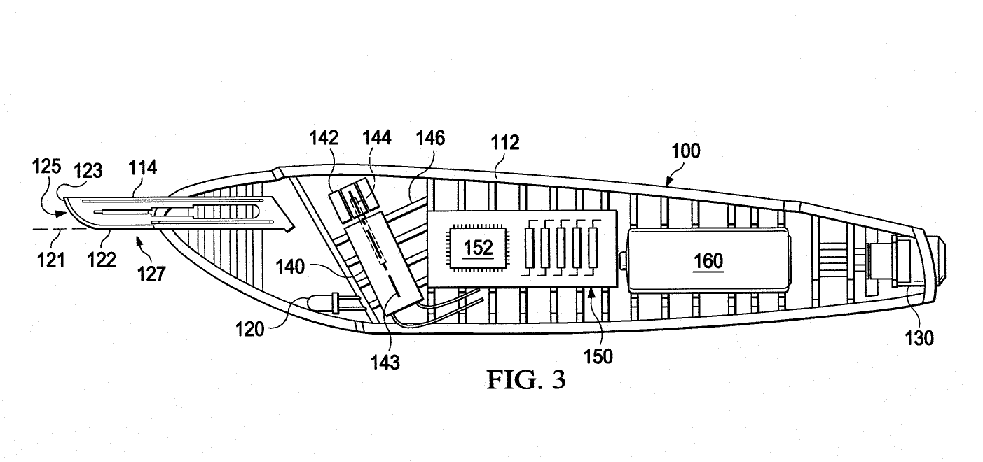

A blade 114, as illustrated in FIGS. 1-3, is attached to or operably restrained by the housing 112 and is used to remove necrotic tissue 104 from the tissue site 110. In some embodiments, the blade 114 may be operably retracted into the housing 112 and extended as needed. The blade 114 may be made of plastic, metal, or any other suitable material. The blade 114 may have smooth, rough, or serrated edges, and may be dull, semi-dull, or sharp. In addition, the blade 114 may consist of various shapes, sizes, and lengths. Selection of a particular type of design of the blade 114 may be based on the type, size, and shape of the tissue site being treated. For example, if the tissue site 110 is a small surface wound which does not require an incision be made for removing the necrotic tissue 104, the blade 114 may be a small, dull flat blade operable to scrape the necrotic tissue 104 away from the tissue site 110 without harming any of the healthy tissue 116 located adjacent the necrotic tissue 104. In some embodiments, the blade 114 may be a regular scalpel blade as illustrated in FIG. 3. Still, in other embodiments, as will be further described, the blade 114 may be a curette-type attachment or a rake shaped blade for removing/scraping the necrotic tissue 104 from the tissue site 110. The blade 114 may be manufactured using an injection molding process or any other suitable method. For example, in certain embodiments, the blade 114 may be a ceramic or plastic injection molded blade.

The debridement tool 100 may also include a light 120 for illuminating the tissue site 110 to aid in debridement of the necrotic tissue 104. The light 120 may be any type of light including a fluorescent light, an incandescent bulb, or light emitting diodes (LEDs). In one embodiment, the light 120 is strategically placed within the housing 112 such that light 120 provides full illumination of the tissue site 110 without casting any shadows onto the tissue site 110. For example, in one embodiment, the light 120 may be placed below a debridement plane 121 (represented by a line in FIG. 3) associated with a debridement edge 122 of the blade 114. In FIG. 3, the debridement plane 121 is perpendicular to the plane of illustration of the figure. The placement of the light 120 beneath the debridement plane 121 allows the light 120 to be between the debridement edge 122 and the tissue being debrided. This positioning helps prevent shadows being cast by the light 120 and provides improved illumination of the tissue. While blade 114 includes a cutting tip 123 and a tip portion 125 of the debridement edge 122 does not follow the debridement plane 121, the debridement edge 122 illustrated in the embodiment of FIG. 3 also includes substantially linear portion 127, and it is upon this portion of the debridement edge 122 that the debridement plane 121 is defined. In other embodiments, a curved edge that does not include linear portions may be provided, making it difficult to definitively identify a debridement plane similar to that illustrated in FIG. 3. In these embodiments, the debridement plane may be defined as corresponding to the average tangential slope associated with the curved cutting edge. Alternatively, instead of the light being positioned beneath the cutting plane or between the cutting plane and the tissue being debrided, the light may be positioned such that the light does not cast a shadow caused by the blade when the blade is positioned in contact with the tissue or above the tissue just prior to contact.

In addition, in certain embodiments, the light 120 may operate at a specific wavelength for aiding the user 102 in distinguishing between the necrotic tissue 104 and the healthy tissue 116 at the tissue site 110. For example, in one embodiment, the light 120 may operate at a red to near infra-red wavelength region, such as, but not limited to, about 500 nm to about 700 nm. In another embodiment, the light may operate at a wavelength of about 500 nm to about 660 nm. In still another embodiment, the wavelength may be about 700 nm. Oxyhemoglobin in the blood stream is capable of absorbing the light at these particular wavelengths, and thus, illuminating the blood vessels within the healthy tissue 116 for distinguishing between the healthy tissue 116 and the necrotic tissue 104. This aids the user 102 in debriding only the necrotic tissue 104 and from inadvertently cutting into the healthy tissue 116.

While the light 120 may be useful to performing debridement operations, all of the embodiments described herein may be used either with or without a light or other illumination source.

Referring still to FIG. 2, in one embodiment, the debridement tool 100 may include a comfortable grip shape 124, a non-slip material or surface 126, and a recharging contact element 128. As stated above, the debridement tool 100 may be ergonomically shaped to provide the user 102 with a comfortable grip so as to aid in debridement of the necrotic tissue 104. The comfortable grip shape 124 may vary in design and may include additional recesses or grooves. In certain embodiments, the comfortable grip shape 124 may be designed specifically for a left hand user or for a right hand user. In addition, the debridement tool 100 may be manufactured in various sizes to accommodate different hand sizes.

The non-slip material or surface 126 prevents slippage of the debridement tool 100 during debridement. For example, the non-slip material or surface 126 may be a rubbery-type material that is wrapped around the housing 112 of the debridement tool 100 for providing a non-slip surface. In addition, the non-slip material or surface 126 may be rough or may include raised ridges or bumps to further prevent slippage of the debridement tool 100. The non-slip material or surface 126 may further include non-slip elements that adhere especially well to latex or rubber gloves to reduce slippage from users that utilize gloves during debridement.

In certain embodiments, the housing 112 contains an internal power source 160, as shown in FIG. 3. The power source 160 may be any type of electrical storage device capable of supplying power to the components of the debridement tool 100. For example, the power source 160 may be, but is not limited to, an alkaline battery, a lithium battery, or a lithium-ion battery.

The recharging contact element 128 enables recharging of the power source 160 within the debridement tool 100. The recharging contact element 128 is electrically coupled to the power source 160 and provides electrons for recharging the power source 160 when in contact with an electrical contact element 132 of a recharging base station 134. The recharging base station 134 is capable of being coupled to an electrical outlet for receiving electricity. The recharging base station 134 may also be used to store the debridement tool 100 when not in use.

Alternatively, the debridement tool 100 may use other recharging techniques for recharging the power source 160 including, but not limited to, inductive coupling or using a recharging port that is operable to receive a plug for recharging the power source 160. Inductive coupling uses magnetic fields that are generated as current moves through a wire. For example, a coil inside the recharging base station 134 creates a magnetic field when current flows through the coil. When the debridement tool 100 is placed on the recharging base station 134, the magnetic field induces a current in another coil within the debridement tool 100, not shown, for recharging the power source 160.

Additionally, in some embodiments, the debridement tool 100 may use a frequency energy receiver 152 coupled to the power source 160 for recharging the power source 160 wirelessly from a remote distance. The frequency energy receiver 152 enables the debridement tool 100 to be recharged by receiving electricity through electromagnetic waves. A wireless energy transmitter, not shown, resonates at the same frequency as the frequency energy receiver 152, and generates the electromagnetic waves. The frequency energy receiver 152 enables the debridement tool 100 to be recharged at a distance of a few meters away from the wireless energy transmitter. In some embodiments, the frequency energy receiver 152 may be integrated with a circuit board 150, as schematically illustrated in FIG. 3. Alternatively, the frequency energy receiver 152 may be a separate component of the debridement tool 100.

Referring to FIG. 3, the circuit board 150 is electrically coupled to the power source 160. The circuit board 150 may include a conductive layer operable to carry power to various components of the debridement tool 100. In one embodiment, the circuit board 150 is coupled to a motor 140, the light 120, and a switch 130 for controlling one or more operations of the debridement tool 100. In another embodiment, the circuit board 150 may be coupled to a blade trigger (not shown) that is operable to detect the position of the blade 204. For example, if the blade trigger detects that the blade is in an extended position, the circuit board 150 may automatically turn on the motor 140, and if the blade trigger detects that the blade is in a retracted position, the circuit board 150 may automatically turn off the motor 140. Similarly, in some embodiments, the debridement tool 100 may include a blade retraction mechanism (not shown) that automatically retracts the blade 114 if the circuit board 150 detects that the motor 140 is powered off, and may automatically extend the blade 114 if the circuit board 150 detects that the motor 140 is powered on.

The switch 130 is used to toggle the motor 140 between an on and off state. In some embodiments, the switch may also activate the light 120. The switch 130 may be a push button switch, a sliding switch, a flip switch, a knob, or any other mechanism for starting and stopping the motor 140. The switch 130 may be placed at any convenient location on the debridement tool 100 such as at the end of the housing 112 (as shown in FIGS. 2 and 3). In another embodiment, not shown, the switch 130 may be placed closer to the blade 114 for enabling a user to easily switch the motor on and off using his/her thumb.

In the depicted embodiment, the motor 140 includes an offset mass 142 that is rotated about an axis 143 associated with a shaft 144. As referenced herein, the term “offset” means that the offset mass 142 is not centrally placed/rotated about the axis 143, is manufactured such that an uneven weight is dispersed about the area of the offset mass 142 (e.g., a first portion of the offset mass 142 weighs more than a second portion of the offset mass 142), or is manufactured with some combination of these characteristics. The rotation of the offset mass 142 about the axis 143 causes vibrations due to the weight imbalance of the offset mass 142. In certain embodiments, the speed at which the offset mass 142 rotates about the axis 143 may be variably controlled to generate a desired level of vibrations. For example, the switch 130 may be a knob that may be gradually adjusted to increase or decrease the level of vibration. In one embodiment, the motor 140 is angled towards the blade 114 for transferring the generated vibrations to the blade 114 for vibrating the blade 114. The size of the motor 140, the offset mass 142, and the blade 114 may vary in different embodiments to provide an optimum level of vibration. The distance between the motor 140 and the blade 114 may also be adjusted to provide the optimum level of vibration. Additionally, the housing 112 may include one or more motor-support mounts 146 for reducing vibrations in undesired planes and for causing the blade 114 to vibrate in a desired plane.

In the embodiment illustrated in FIG. 3, the axis 143 is angled relative to the debridement plane 121 previously described, which results in the rotation of the offset mass 142 generating a first force component substantially parallel to the debridement plane 121 and along a length of the blade 114, a second force component substantially parallel to the debridement plane 121 and substantially transverse to the length of the blade 114, and a third force component substantially perpendicular to the debridement plane 121. These force components assist a user in moving the blade as the user debrides tissue. The rotation of the offset mass 142 results in the oscillation of each force component, which further aids in the debridement of tissue.

In another embodiment, or in any of the embodiments described herein, a motor may be used with to provide a reciprocating motion to the blade or other debridement tip.

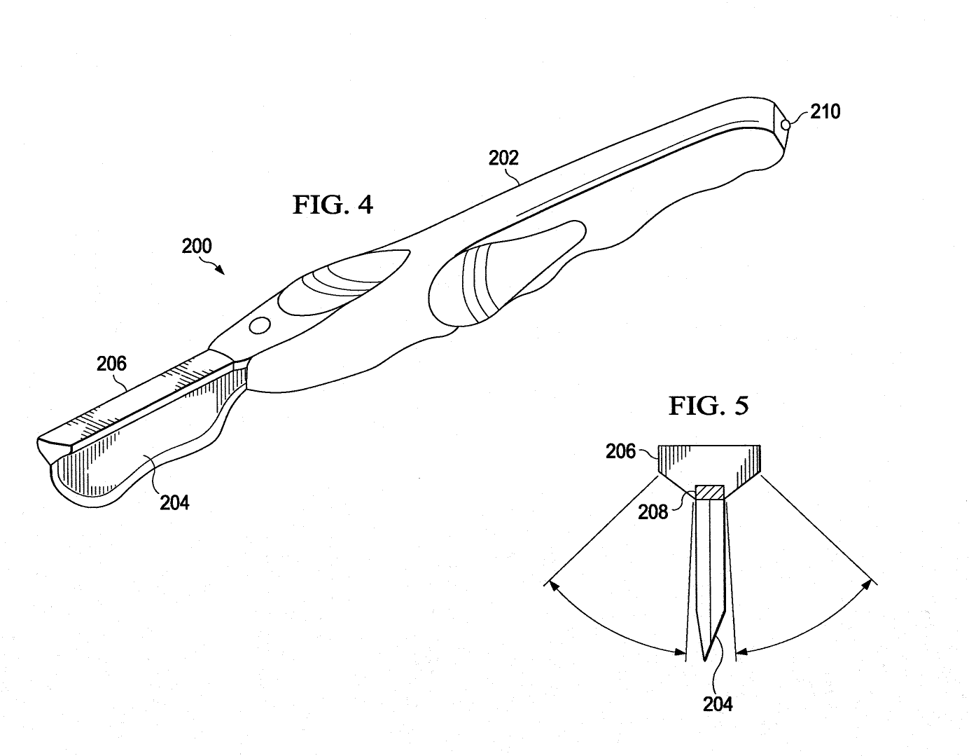

Referring now to FIGS. 4-6, a debridement tool 200 is presented in accordance with another embodiment. FIG. 4 illustrates a perspective view of the exterior of the debridement tool 200. The debridement tool 200 includes a handle 202, a blade 204, and a lighting element 206. The handle 202 may be made of any material including metal, plastic, rubber, or a combination thereof. In a preferred embodiment, the handle 202 is shaped to provide a comfortable grip, and includes a non-slip contact surface. In some embodiments, the debridement tool 200 may include a power plug port 210 for receiving power via an electrical outlet. The power plug port 210 may also be used to recharge an internal power source.

The blade 204 may be any type of blade as described above with regard to the blade 114. For example, the blade 204 may be made of plastic or metal, and may be dull, semi-dull, or sharp. The edge of the blade 204 may be smooth or rough, straight or curved, and may include indentations for enabling easier grasping and debridement of the necrotic tissue 104.

In the depicted embodiment, the lighting element 206 is positioned above the blade 204 for illuminating the tissue site 110. The lighting element 206 may include a recessed groove 208, as shown in FIG. 5, in which the blade 204 resides. The recessed groove 208 is slightly larger than the blade 204 for enabling the blade 204 to vibrate along the axis or width of the groove 208. The lighting element 206 may be operable to illuminate the tissue site 110 on both sides of the blade 204 as illustrated in FIG. 5. By illuminating on both sides of the blade 204, the lighting element 206 is able to illuminate tissue without a shadow being cast by the blade 204. As stated previously, in some embodiments, the lighting element 206 may operate at a specific wavelength for aiding the user 102 in distinguishing between the necrotic tissue 104 and the healthy tissue 116 at the tissue site 110. In one embodiment, the lighting element 206 is fixed to the handle 202 to prevent or reduce the lighting element 206 from vibrating during debridement. Although the depicted lighting element 206 is triangular in form, the lighting element 206 is not limited to any particular shape, size, or length. The housing of the lighting element 206 may be may be made of plastic, metal, or any other suitable material.

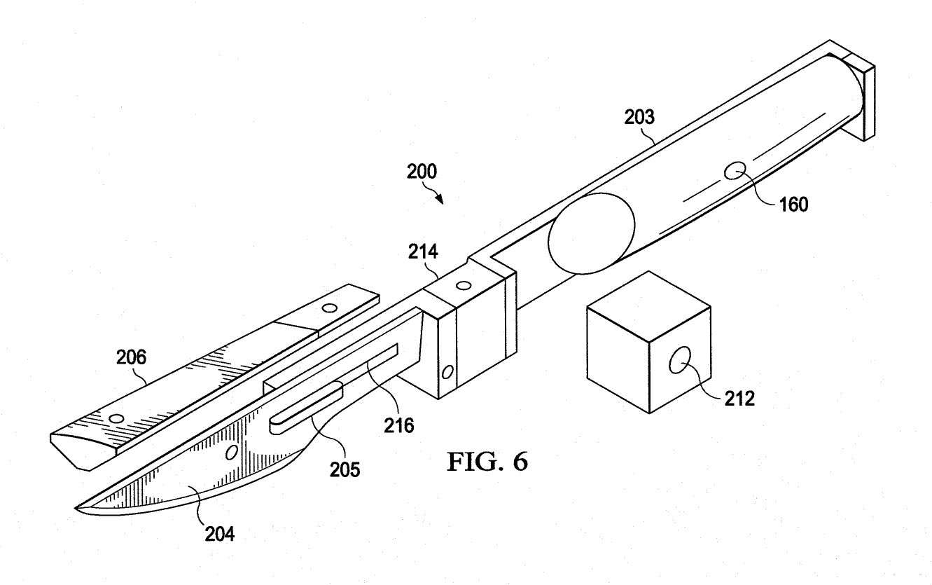

FIG. 6 illustrates components of the debridement tool 200 in accordance with one embodiment. In this embodiment, the debridement tool 200 includes a handle housing 203. The handle housing 203 provides a support structure for coupling the separate components of the debridement tool 200, such as, the power source 160, an electronic controller unit 212, a motor 214, the lighting element 206, and the blade 204. Although the depicted embodiment illustrates a particular arrangement of the components on the handle housing 203, the components may be arranged differently in other embodiments. The handle housing 203 may be made of any suitable material including plastic or metal. In some embodiments, the handle housing 203 may include internal circuitry or wiring for electrically coupling the components of the debridement tool 200.

In certain embodiments, the handle housing 203 may include a blade mount 205 for coupling the blade 204 to the handle housing 203. For example, the blade mount 205 may be a flange that is operable to engage an aperture 216 of the blade 204. In certain embodiments, the blade 204 may be slidingly received on the blade mount 205 such that the blade 204 may reciprocate along the axis of the blade mount 205 due to the vibrations generated by the motor 214. The reciprocating action of blade 204 may aid the user 102 in debriding the necrotic tissue 104. Alternatively, in some embodiments, the blade 204 may be locked or securely fastened to the blade mount 205 to prevent or reduce any reciprocating action to reduce the likelihood of accidentally cutting into the healthy tissue 116 at the tissue site 110.

In the depicted embodiment, the power source 160 is coupled to the handle housing 203 near the end of the handle housing 203. In one embodiment, the power source 160 may be a recharged via the power plug port 210 or by using any other recharging techniques such as, but not limited to, the recharging techniques described above. Alternatively, the power source 160 may be a replaceable power source such as an alkaline or lithium ion battery.

In one embodiment, the electronic controller unit 212 may include circuitry for controlling operations of the motor 214 and the lighting element 206. For example, the electronic controller unit 212 may be coupled to a switch (not depicted) for initiating and stopping the motor 214. In certain embodiments, the electronic controller unit 212 may also house a coil for enabling the power source 160 to be recharged using inductive coupling.

The motor 214 is positioned adjacent the blade 204 along the handle housing 203. The motor 214 generates vibrations that are transferred to the blade 204 for vibrating the blade 204. In some embodiments, the motor is a piezoelectric motor. A piezoelectric motor is a motor that operates by using materials (notably crystals and certain ceramics) to generate vibrations in response to receiving electricity. Alternatively, in some embodiments, the motor 214 may be an ultrasonic motor/transducer, or a sonic or subsonic motor or transducer. An ultrasonic motor is a type of electric motor powered by the ultrasonic vibration of a component, the stator, placed against another component, a rotor or a slider depending on a particular embodiment. The motor 214 may additionally use resonance to amplify the vibration of the stator in contact with the rotor.

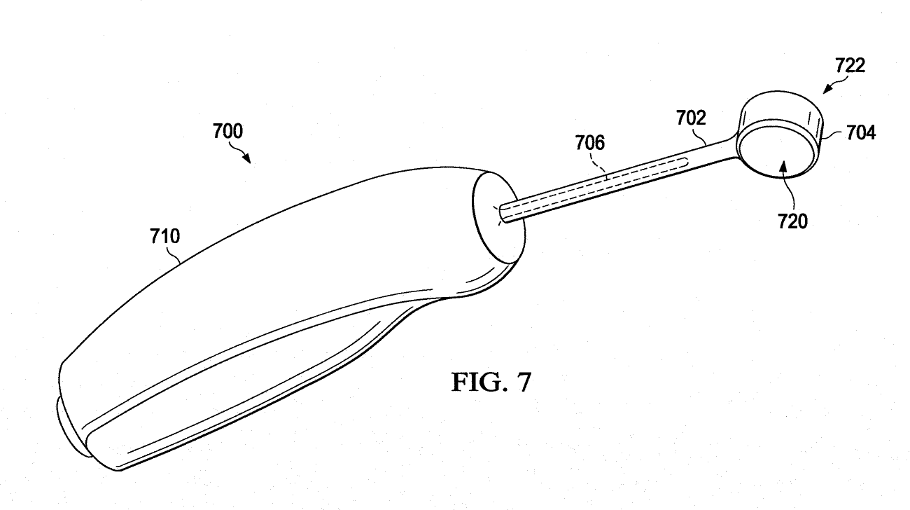

Referring now to FIGS. 7-9, various debridement tips/blades that may be used with the disclosed embodiments for debriding necrotic tissue are presented. Beginning with FIG. 7, a debridement blade 700 that may be used with a debridement tool 710 is presented in accordance with one embodiment. The debridement tool 710 may be one of the disclosed embodiments or may be a generic debridement tool. The debridement blade 700 may be metallic, non-metallic, or partially metallic. In the depicted embodiment, the debridement blade 700 includes an extension member 702, a circular debridement member 704, and an attachment member 706 for coupling the extension member 702 of the blade to the debridement tool 710.

In some embodiments, the extension member 702 may be pliable for enabling the user 102 to bend and shape the extension member 702 as desired for aiding in debridement. In addition, the length and thickness of the extension member 702 may vary in different embodiments.

In one embodiment, the attachment member 706 may be a cavity within the extension member 702 that is operable to couple with a coupling member (not depicted) of the debridement tool 710. The cavity may include threading incorporated along at least a portion of an internal wall for coupling with the coupling member of the debridement tool 710. In an alternative embodiment, the attachment member 706 may include attachment clips (not depicted) operable to couple with a coupling member of the debridement tool. For example, the attachment clips on the extension member 702 may be inserted into an aperture of the debridement tool 710 and locked in place for attaching the debridement blade 700 to the debridement tool 710. Still, in another embodiment, the attachment member 706 may be a male coupling member (not depicted) containing threading for coupling with a female coupling member (not depicted) of the debridement tool 710 (i.e., the debridement blade 700 is twisted onto the debridement tool 710).

The circular debridement member 704 is used to debride necrotic tissue from a tissue site. In one embodiment, the circular debridement member 704 is operable to vibrate due to vibrations generated by the debridement tool 710 to assist a user in debridement. In some embodiments, the circular debridement member 704 is a curette style tip. For instance, the circular debridement member 704 may include an open cavity 720 that is backed by a wall 722 to form a cup-like structure. Alternatively, the circular debridement member 704 may not include the wall 722, but may instead by open to form a ring-like structure. If a cup-like structure is employed, the cup may be cylindrical or domed shaped. The thickness and circumference of the circular debridement member 704 may vary depending on a particular embodiment. For example, the circular debridement member 704 may include a larger circumference or thickness for larger areas of debridement. In certain embodiments, the circular debridement member 704 may include a sharp edge for aiding in debridement of the necrotic tissue 104. Although a circular debridement member is depicted, the circular debridement member 704 is not limited to any particular shape. For example, the circular debridement member 704 may be oval, triangular, diamond, square, rectangular, or any other shape.

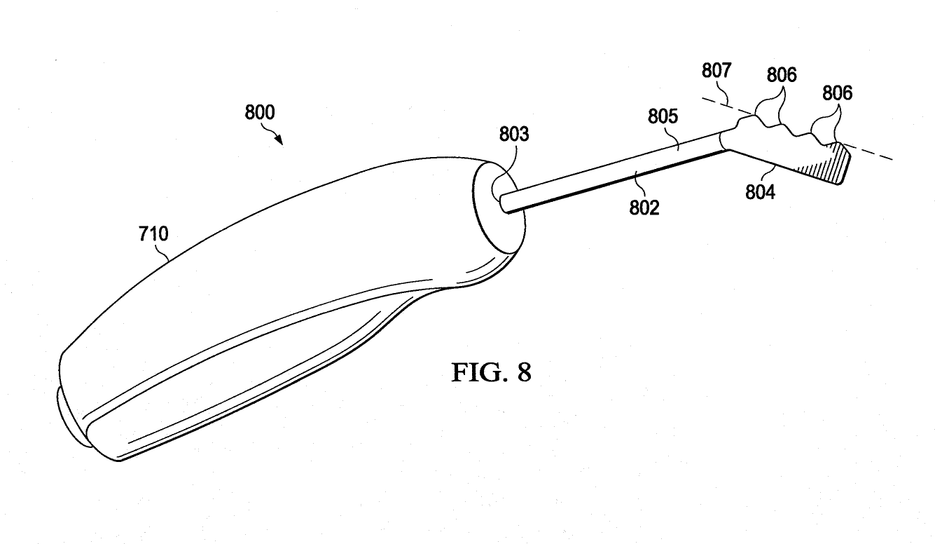

FIG. 8 illustrates another debridement blade 800 that may be used with the debridement tool 710 in accordance with the disclosed embodiments. In the depicted embodiment, the debridement blade 800 includes an extension member 802 having a first end 803 and a second end 805. The debridement blade 800 also includes a debridement member 804 attached to the second end 805 of the extension member.

The extension arm 802 is similar to the extension member 702 as described above with regard to FIG. 7. The extension arm 802 includes an attachment member (not depicted) for coupling the first end 802 of the debridement blade 800 to the debridement tool 710. Alternatively, the extension arm 802 may be connected to or received by the debridement tool 710 without use of a separate attachment member. The attachment member may be the same or similar to that of the attachment member 706 as described above with regard to FIG. 7 or may be any other suitable coupling means. The extension arm 802 may vary in size and length, and may be made of any suitable material.

The debridement member 804 is used to debride necrotic tissue. The debridement member 804 may be metallic, non-metallic (e.g., plastic), or a combination thereof. In one embodiment, the debridement member 804 includes a plurality of ridges 806 that are operable to assist a user in debridement of the necrotic tissue. For example, the plurality of ridges 806 may enable better grasping of necrotic tissue and/or may enable a user to reach into lower areas of a tissue site. In one embodiment, the debridement member 804 is operable to vibrate due to vibrations generated by the debridement tool 710 to assist the user in debridement of necrotic tissue. In some embodiments, the debridement member 804 is coupled to the second end 805 of the extension member 802 such that an angle exists between a debridement plane 807 of the debridement member 804 and a longitudinal axis of the extension member 802. In other words, when angled, the longitudinal axis of the extension member 802 is not parallel to the debridement plane 807. Further, in some embodiments, the debridement member 804 may be adjusted as desired by a user to any particular angle (including parallel) or may be rotatable about the second end 805 of the extension arm 802. Although the depicted embodiment illustrates the second end 805 of the extension arm 802 coupled to one side of the debridement member 804, in some embodiments, the second end 805 of the extension arm 802 may be attached substantially central to the debridement member 804 (i.e., similar to a rake configuration) or to multiple locations of the debridement member 804. In alternative embodiments the debridement member 804 may include a triangular debridement member (not depicted) that is oriented such that a base of the triangular debridement member is used for debridement of necrotic tissue (i.e., similar to a hoe configuration) or oriented such that a tip of the triangular debridement member is used for debridement of necrotic tissue (i.e., an upside down triangle).

FIG. 9 illustrates yet another debridement blade or tip 900 that may be used with the debridement tool 710 in accordance with the disclosed embodiments. The debridement tip 900 is a ribbon-style scraper that comprises an elongated member 904 that forms a shape with an interior space 910. The elongated member 904 is operable to assist a user in debridement of necrotic tissue. For instance, in some embodiments, the elongated member 904 vibrates in response to vibrations generated by the debridement tool 710 in aiding a user in debridement of necrotic tissue.

The elongated member 904 may be made of plastic, rubber, or a composite of any suitable material. Additionally, in certain embodiments, the elongated member 904 may be shaped as desired by a user for providing a greater assortment of angles for enabling the user to debride necrotic tissue at varying tissue sites.

In some embodiments, the debridement tip 900 may include one or more support structures 906 located within the interior space 910 and connected to an interior perimeter 912 of the elongated member 904 for supporting the shape of the elongated member 904. The support structures 906 provide support and prevent the elongated member 904 from collapsing during debridement. The support structures 906 may be rigid or semi-rigid and be made of any suitable material.

The debridement tip 900 may be coupled to the debridement tool 710 via an attachment member (not depicted). The attachment member may be attachment clips or a male coupling similar to the attachment member 706 as described above with regard to FIG. 7. Alternatively, in some embodiments, the debridement tip 900 may be manufactured as an integral part of the debridement tool 710. In addition, in some embodiments, the debridement tip 900 may include an aperture 914 for enabling the light 120 to illuminate a tissue site and for aiding a user in distinguishing between necrotic tissue and healthy tissue.

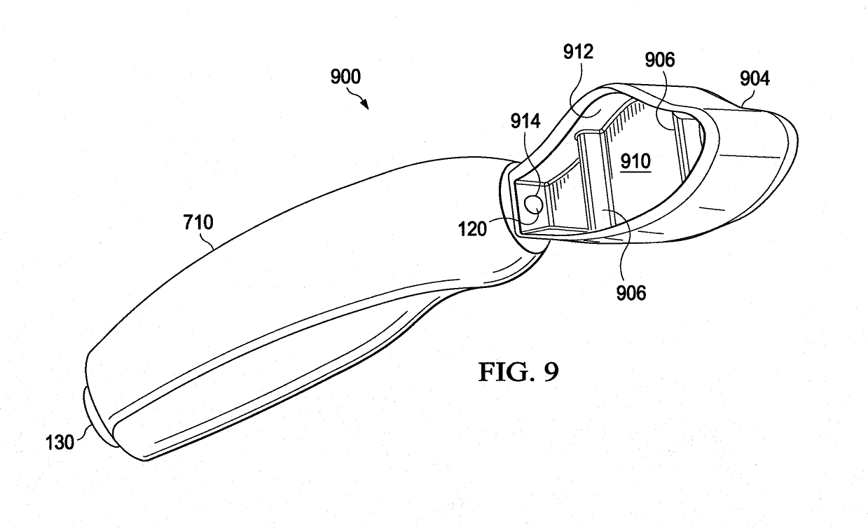

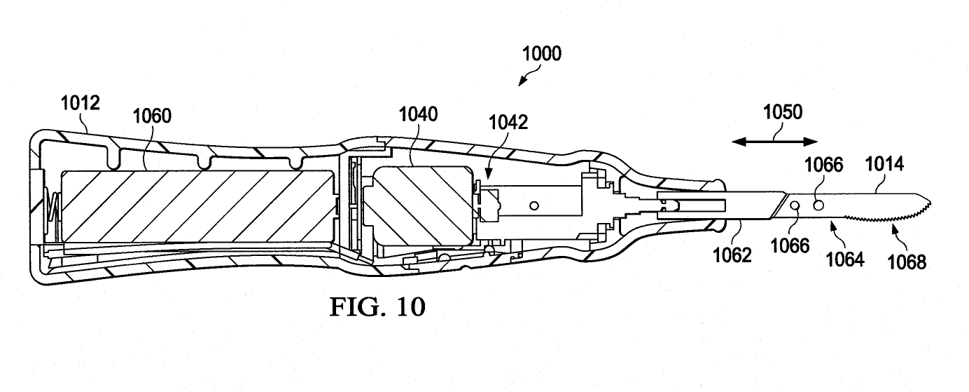

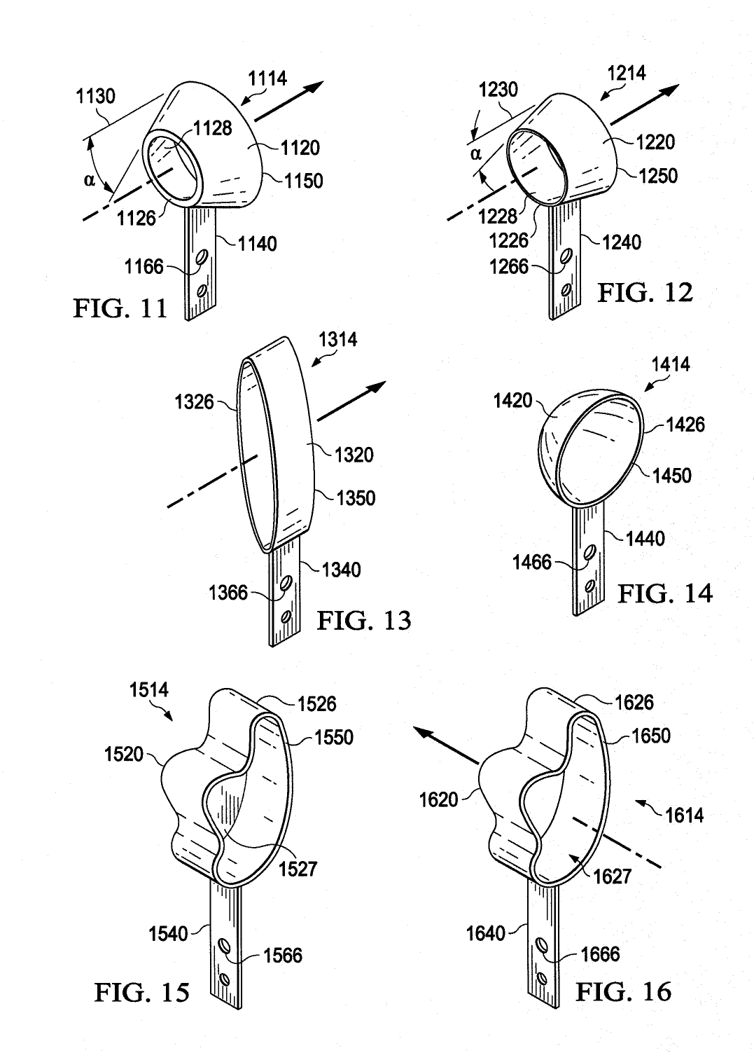

Referring to FIG. 10, a debridement tool 1000 having features similar in some respects to debridement tool 100 is illustrated. Debridement tool 1000 includes a housing 1012 that serves as a handle to allow a user to grip and use the debridement tool 1000. A blade or tip 1014 is received by the housing 1012 and is used to remove necrotic tissue from a tissue site. In some embodiments, the blade 1014 may be operably retracted into the housing 1012 and extended as needed. The blade 1014 may be made of plastic, metal, or any other suitable material. The blade 1014 may have smooth, rough, or serrated edges, and may be dull, semi-dull, or sharp. In addition, the blade 1014 may consist of various shapes, sizes, and lengths. Selection of a particular type of design of the blade 1014 may be based on the type, size, and shape of the tissue site being treated. For example, if the tissue site is a small surface wound which does not require an incision be made for removing the necrotic tissue, the blade 1014 may be a small, dull flat blade operable to scrape the necrotic tissue away from the tissue site without harming any of the healthy tissue located adjacent the necrotic tissue. In some embodiments, the blade 1014 may be a regular scalpel blade. In other embodiments, the blade may be shaped differently, including any of the blades or tips described with reference to FIGS. 11-33.

In certain embodiments, the housing 1012 contains an internal power source 1060, as shown in FIG. 10. The power source 1060 may be any type of electrical storage device capable of supplying power to the components of the debridement tool 1000. For example, the power source 1060 may be, but is not limited to, an alkaline battery, a lithium battery, or a lithium-ion battery. If a battery is used, the battery may employ any suitable charging system to recharge the battery.

The power source 1060 is electrically coupled to a motor 1040 to provide power to the motor 1040. In one embodiment, the motor 1040 is operably coupled to a reciprocating drive train 1042 that may include one or more shafts, gears, or cam. The reciprocating drive train 1042 is capable of imparting reciprocal motion represented by arrows 1050 to a head member 1062. The head member 1062 is coupled to the blade 1014 such that the reciprocating motion of the head member 1062 during operation of the debridement tool 1000 is imparted to the blade 1014. The blade 1014 may include an attachment portion 1064 with a plurality of apertures 1066 or tabs to permit removable coupling to the head member 1062. The blade further includes a debridement portion 1068 with the edges or other components necessary to perform debridement operations.

Referring to FIGS. 11 and 12, debridement blades or tips 1114, 1214 are curette-shaped and each includes a debridement portion 1120, 1220 coupled to an attachment portion 1140, 1240. The debridement portions 1120, 1220 each include a ring-shaped wall 1126, 1226 with at least an inner frusto-conical surface 1128, 1228. In the embodiment illustrated in FIGS. 11 and 12, the outer surface of the ring-shaped wall 1126, 1226 is also frusto-conical. Angle, α, may represent the angle of either the inner surface 1128, 1228 or the outer surface relative to a line 1130 normal to the expected debridement plane, and the angle may in various embodiments be between zero and ninety degrees. A debridement edge 1150, 1250 is provided that may be sharp, dull, smooth, rough, or serrated. The debridement edge 1150, 1250 is capable of removing tissue during debridement operations. The attachment portion of the 1140, 1240 may include a plurality of apertures 1166, 1266 or tabs to permit removable coupling to the head member 1062 (see FIG. 10) or other debridement tools described herein.

Referring to FIG. 13, a debridement blade or tip 1314 is eyelet-shaped and includes a debridement portion 1320 coupled to an attachment portion 1340. The debridement portion 1320 includes a substantially elliptical wall 1326. A debridement edge 1350 is provided that may be sharp, dull, smooth, rough, or serrated. The debridement edge 1350 is capable of removing tissue during debridement operations. The attachment portion of the 1340 may include a plurality of apertures 1366 or tabs to permit removable coupling to the head member 1062 (see FIG. 10) or other debridement tools described herein.

Referring to FIG. 14, a debridement blade or tip 1414 is semi-hemispherical in shape and includes a debridement portion 1420 coupled to an attachment portion 1440. The debridement portion 1420 includes a semi-hemispherical wall 1426. A debridement edge 1450 is provided that may be sharp, dull, smooth, rough, or serrated. The debridement edge 1450 is capable of removing tissue during debridement operations. The attachment portion of the 1440 may include a plurality of apertures 1466 or tabs to permit removable coupling to the head member 1062 (see FIG. 10) or other debridement tools described herein.

Referring to FIG. 15, a debridement blade or tip 1514 is flame-shaped and includes a debridement portion 1520 coupled to an attachment portion 1540. The debridement portion 1520 includes a flame-shaped wall 1526 coupled to a rear wall 1527. A debridement edge 1550 is provided that may be sharp, dull, smooth, rough, or serrated. The debridement edge 1550 is capable of removing tissue during debridement operations. The attachment portion of the 1540 may include a plurality of apertures 1566 or tabs to permit removable coupling to the head member 1062 (see FIG. 10) or other debridement tools described herein.

Referring to FIG. 16, a debridement blade or tip 1614 is flame-shaped and includes a debridement portion 1620 coupled to an attachment portion 1640. The debridement portion 1620 includes a flame-shaped wall 1626 surrounding an inner passage 1627. A debridement edge 1650 is provided that may be sharp, dull, smooth, rough, or serrated. The debridement edge 1650 is capable of removing tissue during debridement operations. The attachment portion of the 1640 may include a plurality of apertures 1666 or tabs to permit removable coupling to the head member 1062 (see FIG. 10) or other debridement tools described herein.

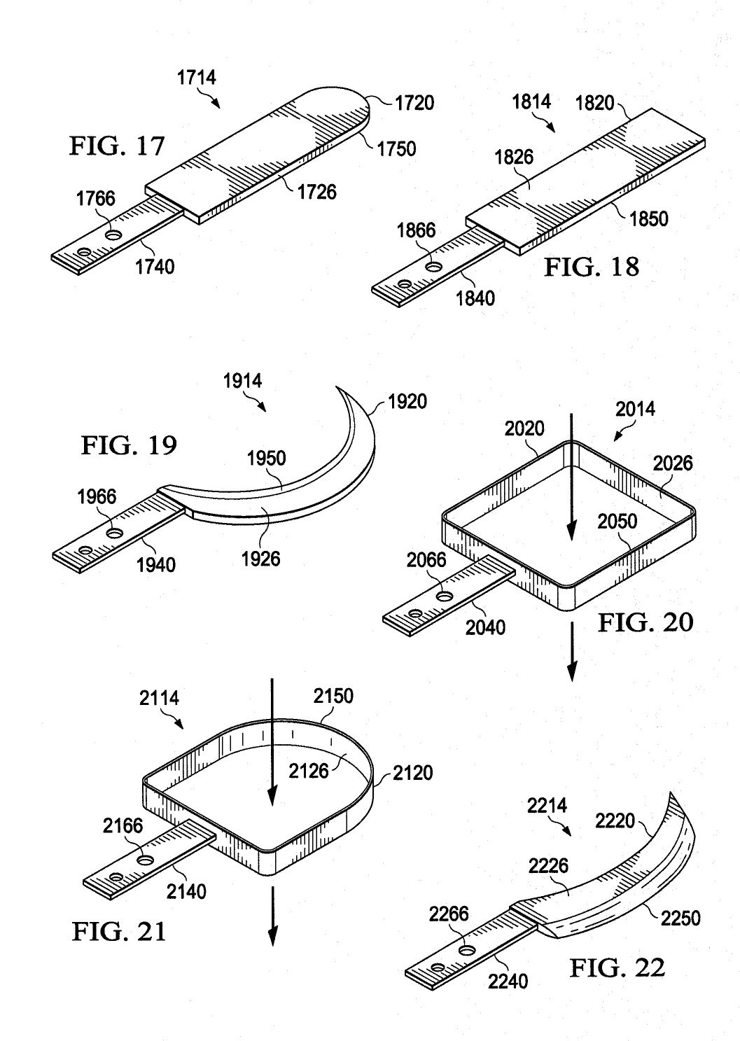

Referring to FIG. 17, a debridement blade or tip 1714 includes a rounded spatula shape with a debridement portion 1720 coupled to an attachment portion 1740. The debridement portion 1720 includes a substantially planar wall 1726. A debridement edge 1750 is provided that may be sharp, dull, smooth, rough, or serrated. The debridement edge 1750 is capable of removing tissue during debridement operations. The attachment portion of the 1740 may include a plurality of apertures 1766 or tabs to permit removable coupling to the head member 1062 (see FIG. 10) or other debridement tools described herein.

Referring to FIG. 18, a debridement blade or tip 1814 includes a squared, spatula shape with a debridement portion 1820 coupled to an attachment portion 1840. The debridement portion 1820 includes a substantially planar wall 1826. A debridement edge 1850 is provided that may be sharp, dull, smooth, rough, or serrated. The debridement edge 1850 is capable of removing tissue during debridement operations. The attachment portion of the 1840 may include a plurality of apertures 1866 or tabs to permit removable coupling to the head member 1062 (see FIG. 10) or other debridement tools described herein.

Referring to FIG. 19, a debridement blade or tip 1914 is sickle-shaped and includes a debridement portion 1920 coupled to an attachment portion 1940. The debridement portion 1920 includes a sickle-shaped wall 1926. A debridement edge 1950 is provided that may be sharp, dull, smooth, rough, or serrated. The debridement edge 1950 is capable of removing tissue during debridement operations. The attachment portion of the 1940 may include a plurality of apertures 1966 or tabs to permit removable coupling to the head member 1062 (see FIG. 10) or other debridement tools described herein.

Referring to FIG. 20, a debridement blade or tip 2014 is square-shaped and includes a debridement portion 2020 coupled to an attachment portion 2040. The debridement portion 2020 includes a square-shaped wall 2026. A debridement edge 2050 is provided that may be sharp, dull, smooth, rough, or serrated. The debridement edge 2050 is capable of removing tissue during debridement operations. The attachment portion of the 2040 may include a plurality of apertures 2066 or tabs to permit removable coupling to the head member 1062 (see FIG. 10) or other debridement tools described herein.

Referring to FIG. 21, a debridement blade or tip 2114 is square-shaped and includes a debridement portion 2120 coupled to an attachment portion 2140. The debridement portion 2120 includes a square-shaped wall 2126 that includes a pair of rounded corners. A debridement edge 2150 is provided that may be sharp, dull, smooth, rough, or serrated. The debridement edge 2150 is capable of removing tissue during debridement operations. The attachment portion of the 2140 may include a plurality of apertures 2166 or tabs to permit removable coupling to the head member 1062 (see FIG. 10) or other debridement tools described herein.

Referring to FIG. 22, a debridement blade or tip 2214 is tanto-shaped and includes a debridement portion 2220 coupled to an attachment portion 2240. The debridement portion 2220 includes a tanto-shaped wall 2226. A debridement edge 2250 is provided that may be sharp, dull, smooth, rough, or serrated. The debridement edge 2250 is capable of removing tissue during debridement operations. The attachment portion of the 2240 may include a plurality of apertures 2266 or tabs to permit removable coupling to the head member 1062 (see FIG. 10) or other debridement tools described herein.

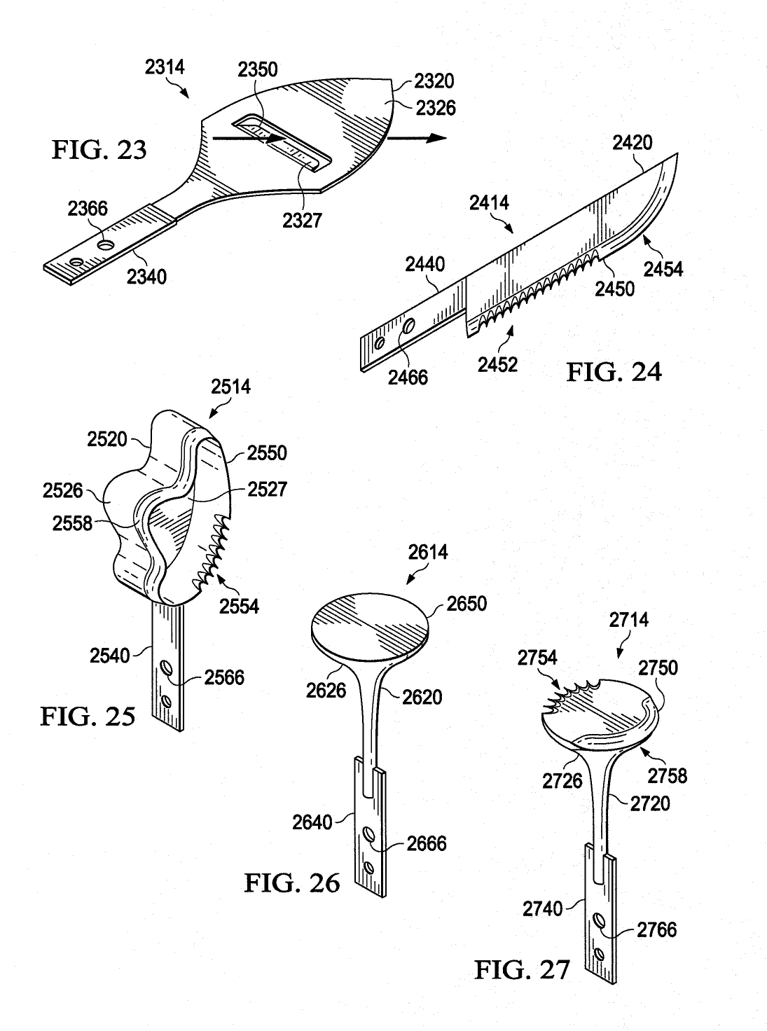

Referring to FIG. 23, a debridement blade or tip 2314 is spade-shaped and includes a debridement portion 2320 coupled to an attachment portion 2340. The debridement portion 2320 includes a spade-shaped wall 2326 and includes a debridement element 2327 that is angled relative to the wall 2326. A debridement edge 2350 is provided that may be sharp, dull, smooth, rough, or serrated. The debridement edge 2350 is capable of removing tissue during debridement operations. The attachment portion of the 2340 may include a plurality of apertures 2366 or tabs to permit removable coupling to the head member 1062 (see FIG. 10) or other debridement tools described herein.

Referring to FIG. 24, a debridement blade or tip 2414 includes a debridement portion 2420 coupled to an attachment portion 2440. The debridement portion 2420 includes a debridement edge 2450 that includes both a linear region 2452 and a curved region 2454. The debridement edge 2450 in the linear region 2452 is serrated. The curved region 2454 is non-serrated, but sharpened. It should be noted that the linear and curved regions each may include any combination of sharp, dull, smooth, rough, or serrated edges. The debridement edge 2450 is capable of removing tissue during debridement operations. The attachment portion of the 2440 may include a plurality of apertures 2466 or tabs to permit removable coupling to the head member 1062 (see FIG. 10) or other debridement tools described herein.

Referring to FIG. 25, a debridement blade or tip 2514 is flame-shaped and includes a debridement portion 2520 coupled to an attachment portion 2540. The debridement portion 2520 includes a flame-shaped wall 2526 coupled to a rear wall 2527. A debridement edge 2550 is provided that in FIG. 25 includes a coarsely serrated region 2554 and a sharpened region 2558. The debridement edge 2550 is capable of removing tissue during debridement operations. The attachment portion of the 2540 may include a plurality of apertures 2566 or tabs to permit removable coupling to the head member 1062 (see FIG. 10) or other debridement tools described herein.

Referring to FIG. 26, a debridement blade or tip 2614 is valve-shaped and includes a debridement portion 2620 coupled to an attachment portion 2640. The debridement portion 2620 includes a valve-shaped member 2626. A debridement edge 2650 is provided that may be sharp or non-sharpened (i.e. dull). The debridement edge 2650 is capable of removing tissue during debridement operations. The attachment portion of the 2640 may include a plurality of apertures 2666 or tabs to permit removable coupling to the head member 1062 (see FIG. 10) or other debridement tools described herein.

Referring to FIG. 27, a debridement blade or tip 2714 is valve-shaped and includes a debridement portion 2720 coupled to an attachment portion 2740. The debridement portion 2720 includes a valve-shaped member 2726. A debridement edge 2750 is provided that includes a coarsely serrated region 2754 and a sharpened region 2758. The debridement edge 2750 is capable of removing tissue during debridement operations. The attachment portion of the 2740 may include a plurality of apertures 2766 or tabs to permit removable coupling to the head member 1062 (see FIG. 10) or other debridement tools described herein.

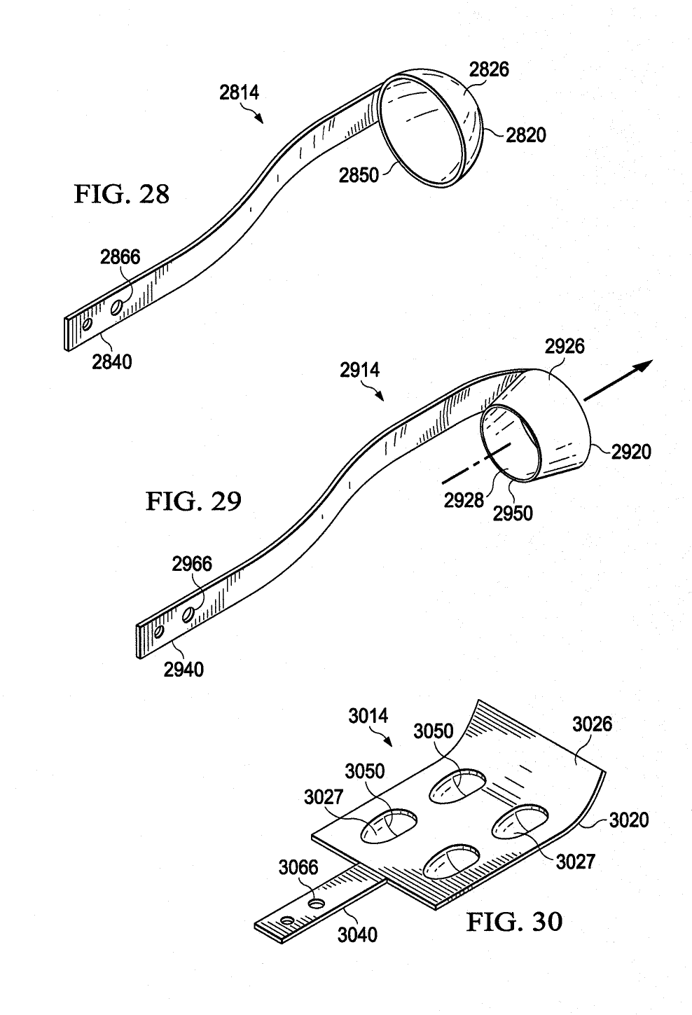

Referring to FIG. 28, a debridement blade or tip 2814 is semi-hemispherical in shape and includes a debridement portion 2820 coupled to an attachment portion 2840. The attachment portion 2840 includes an angled region that allows the debridement portion 2820 to be oriented differently than the debridement portion 1420 of FIG. 14. In FIG. 28, the debridement portion 2820 is oriented with its opening directed toward the debridement tool. The debridement portion 2820 includes a semi-hemispherical wall 2826. A debridement edge 2850 is provided that may be sharp, dull, smooth, rough, or serrated. The debridement edge 2850 is capable of removing tissue during debridement operations. The attachment portion of the 2840 may include a plurality of apertures 2866 or tabs to permit removable coupling to the head member 1062 (see FIG. 10) or other debridement tools described herein.

Referring to FIG. 29, a debridement blade or tip 2914 is curette-shaped and includes a debridement portion 2920 coupled to an attachment portion 2940. The attachment portion 2940 includes an angled region that allows the debridement portion 2920 to be oriented differently than the debridement portion 1120 of FIG. 11. In FIG. 29, the debridement portion 2920 is oriented with its opening directed toward the debridement tool. The debridement portion 2920 includes a ring-shaped wall 2926 with at least an inner frusto-conical surface 2928 similar to the debridement portion 1120 of FIG. 11. A debridement edge 2950 is provided that may be sharp, dull, smooth, rough, or serrated. The debridement edge 2950 is capable of removing tissue during debridement operations. The attachment portion of the 2940 may include a plurality of apertures 2966 or tabs to permit removable coupling to the head member 1062 (see FIG. 10) or other debridement tools described herein.

Referring to FIG. 30, a debridement blade or tip 3014 is sled-shaped and includes a debridement portion 3020 coupled to an attachment portion 3040. The debridement portion 3020 includes a rectangular, sled-shaped wall 3026 and includes a plurality of debridement elements 3027 that protrude beneath the wall 3026. A debridement edge 3050 is provided that may be sharp, dull, smooth, rough, or serrated. The debridement edge 3050 is capable of removing tissue during debridement operations. The attachment portion of the 3040 may include a plurality of apertures 3066 or tabs to permit removable coupling to the head member 1062 (see FIG. 10) or other debridement tools described herein.

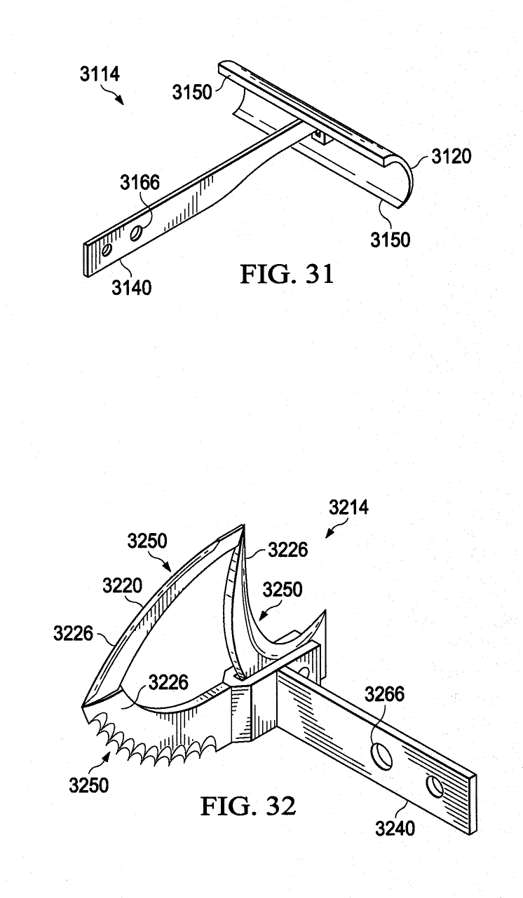

Referring to FIG. 31, a debridement blade or tip 3114 is plow-shaped and includes a debridement portion 3120 coupled to an attachment portion 3140. The debridement portion 3120 includes a rectangular, plow-shaped member 3126. At least one debridement edge 3150 is provided that may be sharp, dull, smooth, rough, or serrated. The debridement edge 3150 is capable of removing tissue during debridement operations. The attachment portion of the 3140 may include a plurality of apertures 3166 or tabs to permit removable coupling to the head member 1062 (see FIG. 10) or other debridement tools described herein.

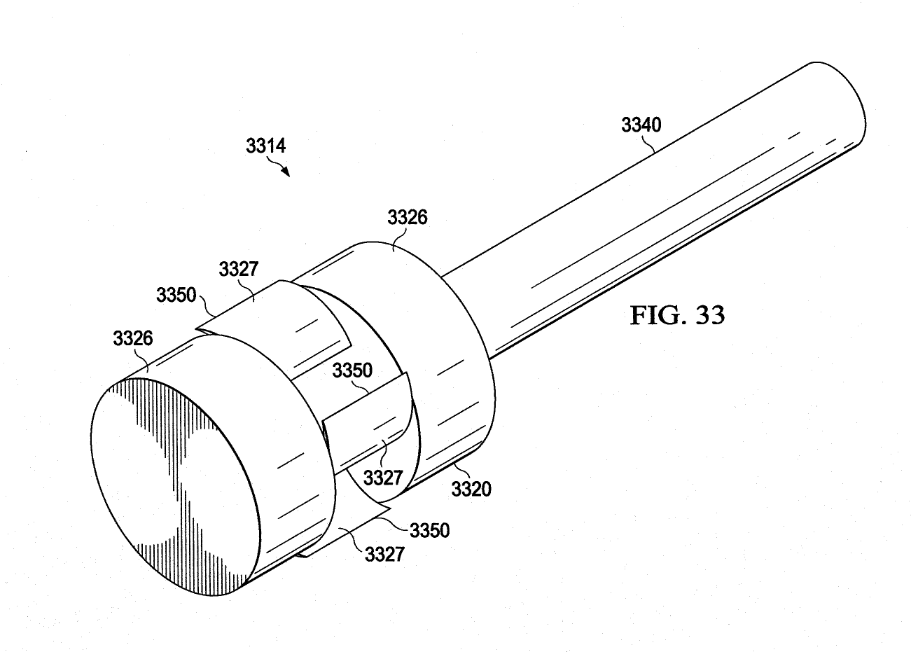

Referring to FIG. 32, a debridement blade or tip 3214 includes a debridement portion 3220 coupled to an attachment portion 3240. The debridement portion 3220 includes a plurality of curved members 3226. At least one debridement edge 3250 is provided that may be sharp, dull, smooth, rough, or serrated. The debridement edge 3250 is capable of removing tissue during debridement operations. The attachment portion of the 3240 may include a plurality of apertures 3266 or tabs to permit removable coupling to the head member 1062 (see FIG. 10) or other debridement tools described herein.

Referring to FIG. 33, a debridement blade or tip 3314 is cylindrically-shaped and includes a debridement portion 3320 coupled to an attachment portion 3340. The debridement portion 3320 includes a pair of cylindrically-shaped walls 3326 between which are located a plurality of debridement elements 3327 that radially protrude beyond the cylindrically-shaped walls 3326. A debridement edge 3350 is provided on each debridement element 3327 that may be sharp, dull, smooth, rough, or serrated. The debridement edge 3350 is capable of removing tissue during debridement operations. The attachment portion of the 3340 of FIG. 33 is preferably a shaft that is configured to be rotated by a motor or other motion-imparting device. As the shaft is rotated, the debridement elements 3327 also rotate thereby allowing efficient removal of tissue. The presence of the cylindrically-shaped walls 3326 provides safe operation by limiting the depth to which the debridement elements 3327 may remove tissue. In other words, the debridement elements 3327 are only capable of removing tissue to a depth equal to the distance that the debridement elements 3327 extend beyond the walls 3326. This distance is preferably adjustable to allow adjustability in the depth of tissue removal.

Accordingly, based on the above disclosure, it should be apparent from the foregoing that an invention having significant advantages has been provided for aiding a user in debriding necrotic tissue. While the depicted embodiments illustrate certain shapes and design configurations, the disclosed embodiments are not intended to be limited to any particular design, and are susceptible to various changes and modifications without departing from the spirit thereof.

1. A debridement tool for debriding tissue from a tissue site, the debridement tool comprising:
a housing having an interior space;
a motor disposed within the interior space of the housing;
a power source disposed within the interior space of the housing operable to provide power to the motor;
a blade coupled to a first end of the housing, the blade operable to vibrate when the motor is powered on; and
a lighting element disposed within the housing and beneath a debridement edge of the blade, the lighting element operable to illuminate the tissue site.

2. The debridement tool of claim 1, further comprising a switch for powering the motor on and off.

3. The debridement tool of claim 2, wherein the switch is located at a second end of the housing, the second end being opposite the first end.

4. The debridement tool of claim 1, wherein the power source is a rechargeable power source operable to be recharged while remaining in the housing.

5. The debridement tool of claim 1, wherein the power source is a rechargeable power source operable to be recharged using inductive coupling.

6-9. (canceled)

10. The debridement tool of claim 1, wherein the motor is a piezoelectric motor.

11. The debridement tool of claim 1, wherein the motor is an ultrasonic motor.

12. The debridement tool of claim 1, wherein the motor includes an offset mass rotating about an axis causing the blade to vibrate.

13. The debridement tool of claim 12, wherein the axis is angled relative to a debridement plane of the blade such that the rotation of the offset mass generates a first force component substantially parallel to the debridement plane and along the length of the blade, a second force component substantially parallel to the debridement plane and substantially transverse to the length of the blade, and a third force component substantially perpendicular to the debridement plane.

14. The debridement tool of claim 13, further comprising one or more mounts within the housing for reducing the vibration in undesired planes caused by the rotation of the offset mass.

15. The debridement tool of claim 1, wherein the blade is unsharpened.

16. The debridement tool of claim 1, wherein the vibration of the blade assist in debridement of the tissue.

17. The debridement tool of claim 1, wherein the blade is a metal scalpel blade.

18. (canceled)

19. The debridement tool of claim 1, wherein the blade is an injection molded plastic blade.

20. The debridement tool of claim 1, wherein the blade is an injection molded ceramic blade.

21. The debridement tool of claim 1, wherein the blade vibrates at ultrasonic frequencies.

22. The debridement tool of claim 1, wherein the blade is retractable.

23. The debridement tool of claim 22, further comprising a blade trigger, the blade trigger causing the motor to power off when the blade is retracted, and causing the motor to power on when the blade is extended.

24. The debridement tool of claim 1, wherein the shape of the blade is dependent on the tissue site being treated.

25. The debridement tool of claim 1, wherein the lighting element is operable to identify tissue with blood flow.

26. The debridement tool of claim 1, wherein the lighting element is operable to identify necrotic tissue.

27. The debridement tool of claim 1, wherein the lighting element operates at a specific wavelength for identifying tissue with blood flow.

28. The debridement tool of claim 27, wherein the specific wavelength is at a red to near infra-red region.

29. The debridement tool of claim 1, further comprising electrical circuitry electrically coupled to the motor, the power source, and the lighting element for controlling operations of the debridement tool.

30-68. (canceled)

69. A debridement tool for debriding tissue from a tissue site, the debridement tool comprising:
a housing having an interior space;
a motor disposed within the interior space of the housing, wherein the motor includes an offset mass rotating about an axis for generating vibrations;
a power source disposed within the interior space of the housing operable to provide power to the motor;
a blade coupled to a first end of the housing along a first plane, the motor located a distance and angled within the housing so as to transfer the vibrations along the first plane for vibrating the blade along the first plane for assisting a user in debridement of necrotic tissue; and
a lighting element disposed within the housing beneath a debridement edge of the blade, wherein the lighting element operates at a wavelength operable to assist a user in distinguishing between necrotic tissue and healthy tissue.
